(19) United States
(12) Patent Application Publication
(10) Pub. No.: US 2012/0310351 A1
(43) Pub. Date: Dec. 6, 2012

(54) LORDOTIC SPACER

(76) Inventor: Daniel K. Farley, Traverse City, MI (US)

(21) Appl. No.: 13/488,009

(22) Filed: Jun. 4, 2012

Related U.S. Application Data
(60) Provisional application No. 61/492,577, filed on Jun. 2, 2011.

(51) Int. Cl. A61F 2/44

(52) U.S. Cl. 623/17.16

(57) ABSTRACT

A spacer includes a distal end and a proximal end. The spacer also includes top and bottom surfaces spaced by first and second sides. The top and bottom surfaces define a height, and the sides define a width. The height generally increases distally and from the second side to the first side. The top and bottom surfaces are curved, and include multiple radii of curvature. A lateral curvature is defined as the curvature along the top or bottom surface at a given point along the length of the spacer, taken along a plane extending between the first and second sides and generally perpendicular to the sides. The radius of the lateral curvature for at least one of the top and bottom sides varies along the length of the spacer. For example, the radius of curvature may generally increase distally along the length of the spacer.

### CROSS-REFERENCE TO RELATED APPLICATION

None.

### STATEMENT REGARDING FEDERALLY SPONSORED RESEARCH OR DEVELOPMENT

None.

### BACKGROUND OF THE INVENTION

The present invention relates to systems and methods for providing spinal implants, for example, to be used in connection with spinal fusion for correction and/or treatment of an improper curvature, or lordosis, of the spine.

Spinal fusion is a surgical procedure that fuses two or more vertebrae together using bone graft materials supplemented with devices. Spinal fusion may be performed for the treatment of chronic neck and/or back pain, trauma, and neoplasms. Spinal fusion can be used to stabilize and eliminate motion of vertebrae segments that may be unstable, or move in an abnormal way, that can lead to discomfort and pain. Spinal fusion may be performed to treat injuries to the vertebrae, degeneration of spinal discs, abnormal spinal curvature, and/or a weak or unstable spine.

Lordosis refers to a curvature of the spine, specifically a curvature that is posteriorly concave in the lumbar region of the spine. In certain patients, this curvature may, for example, be larger than desired. When aligning the spine during a fusion of lumbar vertebrae, it is desirable to properly set the curvature. However, many known spacer designs for use in spinal fusion do not account for this curvature as well as desired. Further, many do not match the surface of the vertebrae being fused as closely as desired, requiring increased time for preparing the vertebral surfaces and/or removal of increased amount of bone from the spinal column to accept and/or match a spacer to be inserted.

Spinal fusion generally requires a graft material, usually bone material, to fuse the vertebrae together. The bone graft material can be placed over the spine to fuse adjacent vertebrae together. Alternatively, a device (i.e. cage) may be positioned between the vertebrae being fused and filled with the bone graft material. Such a cage can include holes that allow the vertebrae and the graft material to grow together to provide fusion, with the cage supporting the weight of the vertebrae while the fusion is occurring. Most of these cages are limited to only a few cubic centimeters of bone graft material thus limiting the fusion area achieved. Because the fusion mass is under pressure, fusion can be promoted. The disc space height can be restored, taking pressure off of the nerves. The spine alignment, foraminal height, and canal diameter can be restored. In some cases the graft can be placed with minimal disruption of muscles and ligaments using minimally invasive approaches to the spine, thus preserving the normal anatomical integrity of the spine. Other interbody device assemblies are also presently known. These include those disclosed in U.S. patent application Ser. Nos. 11/623,356, filed Jan. 16, 2007, titled “Minimally Invasive Interbody Device,” and 11/932,175, filed Oct. 31, 2007, titled “Minimally Invasive Interbody Device Assembly,” which are hereby incorporated by reference in their entirety.

Typically, the bone graft material is autogenous bone material taken from the patient, or allograft bone material harvested from a cadaver. Synthetic bone material can also be used as the graft material. Generally, the patient's own bone material offers the best fusion material since it offers osteoinductive, osteoconductive, and osteogenesis properties. Known bone fusion materials include iliac crest harvest from the patient, bone graft extenders, such as hydroxyapetite and demineralized bone matrix, and bone morphogenic protein.

It is therefore one object of the present invention to provide a spinal implant system that provides improved control over the curvature of the spine, and/or provides for improved insertion of a spacer to be used for spinal fusion.

### BRIEF SUMMARY OF THE INVENTION

These and other objects of the invention are achieved in a spinal implant, or spacer. The spacer includes a distal end and a proximal end. The spacer also includes top and bottom surfaces spaced by first and second sides. The top and bottom surfaces define a height, and the sides define a width. The height generally increases distally and from the second side to the first side. In certain embodiments, the height is greater than the width, wherein the spacer may be inserted with its sides oriented toward surfaces of adjacent vertebrae and then rotated into place with the top and bottom surface oriented toward the surfaces of the adjacent vertebrae to maintain a desired space between the adjacent vertebrae. The top and bottom surfaces are curved, and include multiple radii of curvature. A lateral curvature is defined as the curvature along the top or bottom surface at a given point along the length of the spacer, taken along a plane extending between the first and second sides and generally perpendicular to the sides. The radius of the lateral curvature for at least one of the top and bottom sides varies along the length of the spacer. For example, the radius of curvature may generally increase distally along the length of the spacer.

### BRIEF DESCRIPTION OF THE SEVERAL VIEWS OF THE DRAWINGS

FIG. 1 illustrates a perspective view of a lordotic spacer formed in accordance with an embodiment of the present invention.

FIG. 2 illustrates a plan view of the lordotic spacer of FIG. 1.

FIG. 3 illustrates a side view of the lordotic spacer of FIG. 1.

FIG. 4 illustrates an end view of the lordotic spacer of FIG. 1.

FIG. 5 illustrates a sectional view of the lordotic spacer of FIG. 3 taken along line 5-5.

FIG. 6 illustrates a sectional view of the lordotic spacer of FIG. 3 taken along line 6-6.

FIG. 7 illustrates a sectional view of the lordotic spacer of FIG. 3 taken along line 7-7.

FIG. 8 illustrates a plan view of a lordotic spacer formed in accordance with an embodiment of the present invention in position in a patient.

### DETAILED DESCRIPTION

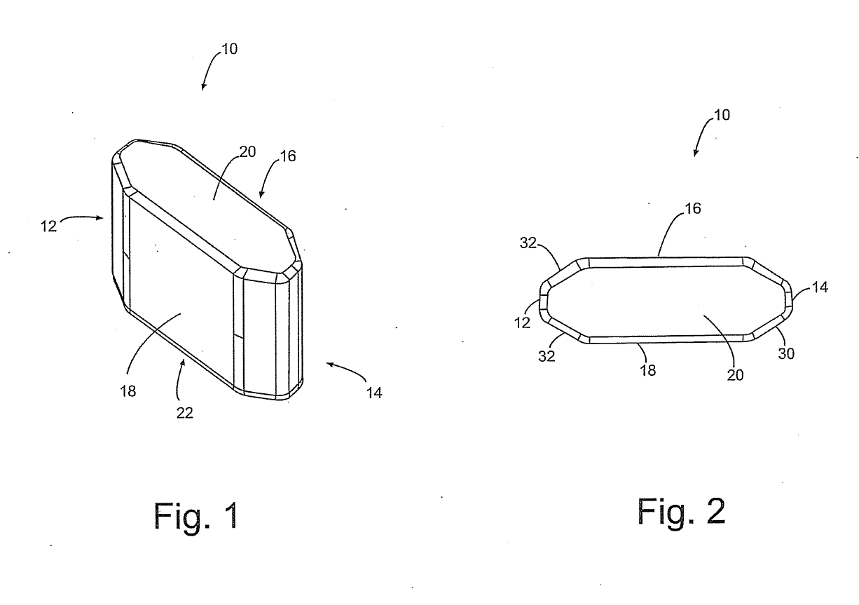

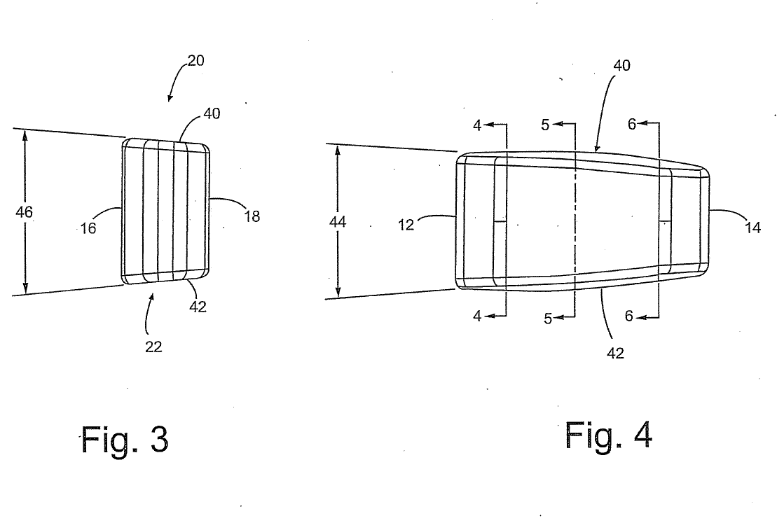

FIG. 1 illustrates a perspective view of a lordotic spacer 10 formed in accordance with an embodiment of the present invention. FIG. 2 illustrates a plan view of the lordotic spacer 10, FIG. 3 illustrates a side view of the lordotic spacer 10, and FIG. 4 illustrates an end view of the lordotic spacer 10. The illustrated lordotic spacer 10 is particularly well adapted to be inserted at an angle (i.e. not substantially aligned with a line generally extending directly anteriorly to posteriorly and not substantially aligned with a line extending directly laterally from side to side) into the spine to help correct a lordosis. For example, the lordotic spacer may, in some embodiments, be configured to be inserted into a patient at about 45 degrees to a line drawn in a posterior-anterior orientation (which would also be at about 45 degrees to a line drawn in a lateral side to side direction). Different angles may be used for different embodiments and/or different patient and/or procedures. The lordotic spacer includes a distal end 12, a proximal end 14, a first side 16, a second side 18, a top 20, and a bottom 22.

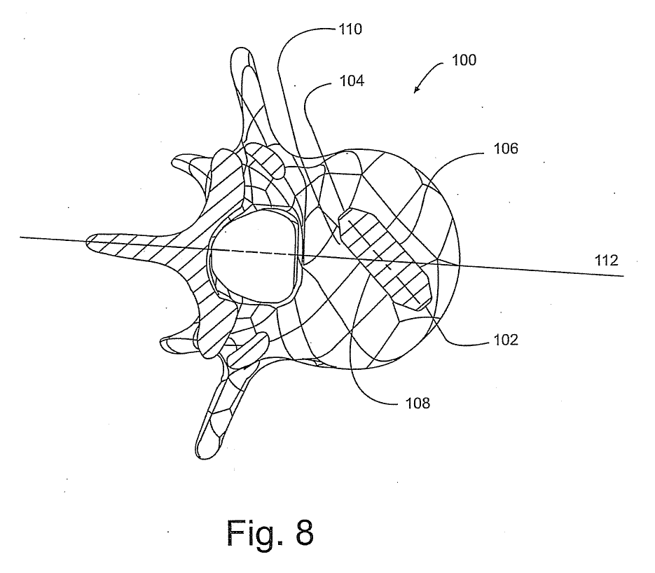

The distal end 12 is the end of the lordotic spacer 10 that is designed to be inserted more deeply into a patient during a procedure, while the proximal end 14 is the end designed to be oriented closer to the practitioner (or closer to the surface of the patient's body) when inserted in place. In the illustrated embodiment, the height (the distance between the top and bottom) of the lordotic spacer generally increases distally, so that the lordotic spacer 10 can help re-align or counteract an undesirable amount of lordosis previously present in a patient's spine, and/or help maintain a correct amount of lordosis. Further, the lordotic spacer 10 is configured to be placed at an angle in the patient. Put another way, the lordotic spacer 10 is configured so that one of its lateral sides is oriented generally more distally than the other of its lateral sides. In the illustrated embodiment, the first side 16 has a generally greater height than the second side 18, and the first side 16 is configured so that it is positioned generally distally of the second side when the lordotic spacer is in place in a patient (see also FIG. 8 and related discussion). Thus, the relative heights of the distal end and the first side (generally having greater heights than the proximal end and the second side, respectively), help to maintain a posterior concavity when the lordotic spacer 10 is in place in a patient. The difference between the heights of the sides and/or ends are selected to help provide the proper concavity.

Further, the height of the lordotic spacer 10 may be generally larger than its width. This allows the lordotic spacer 10 to be inserted between the vertebrae of interest sideways and then rotated to distract and maintain the vertebrae in their desired relationship. In the illustrated embodiments, the top 20 and bottom 22 are depicted as generally smooth surfaces. In alternate embodiments, the top and/or the bottom, and/or portions of the top and bottom, may include projections, grooves, or other features to help secure the spacer in place. Yet further still, in certain embodiments, the spacer may include cutouts, projections or other features to assist in grasping and/or holding the spacer during insertion and manipulation, and/or to assist in distributing or guiding bone graft material around the spacer once it is inserted in place. Further, in certain embodiments, the spacer may contain hollow portions, cutouts, channels, conduits or the like to facilitate the insertion of bone graft material into and/or through the spacer.

FIG. 2 illustrates a plan view of the lordotic spacer 10. As can be seen in FIG. 2, the lordotic spacer includes proximal tapers 30 and distal tapers 32. The tapers are tapered sections of reduced width tapering toward the ends, such as the distal end 12 or the proximal end 14. The distal tapers 32 may be sized and configured to ease insertion and/or rotation of the lordotic spacer 10, while the proximal tapers 30 may be sized and configured to facilitate grasping of the lordotic spacer 10 and/or to facilitate distribution of bone graft material to either side of the lordotic spacer 10. In other embodiments, there may be only tapers proximal to only one of the distal and proximal ends, or there may be no tapers at all. In the illustrated embodiment, the proximal tapers 30 and distal tapers 32 are proportioned substantially similarly. In alternate embodiments, the proximal and distal tapers could be sized and configured differently from each other.

FIG. 3 illustrates a side view of the lordotic spacer 10. As seen in FIG. 3, the top 20 of the lordotic spacer includes a top curved surface 40, and the bottom 22 of the lordotic spacer includes a bottom curved surface 42. As also discussed below, these surfaces may be formed to include various radii. For example, in the illustrated embodiment, the top curved surface 40 presents a generally uniform radius along its length (the distance from the distal end 12 to the proximal end 14) when viewed from the side. For example, the length of the lordotic spacer 10 may about 30 millimeters, and the radius, when viewed from the side, may be about 130.1 millimeters. The bottom curved surface 42 may be configured substantially similarly to the top curved surface 40, or, in alternate embodiments, the bottom curved surface 42 may have different proportions, such as radius when viewed from the side, than the top curved surface 40. In other embodiments, the radius may not be the same throughout, or the surface could include angled and/or stepped portions in addition or alternatively to the radius when viewed from the side.

As can also be seen in FIG. 3, the lordotic spacer 10 generally increases in height distally. For example, tangent lines to the top curved surface 40 and bottom curved surface 42 in a generally proximal to distal direction may diverge at an angle 44. In the illustrated embodiment, the angle 44 is about 6 degrees. While the lordotic spacer 10 generally increases in height (the distance between the top and bottom surfaces) distally, the maximum height may not necessarily be at the distal end 12 due to the radiusing, but may instead be at a distal portion of the spacer near the distal end 12.

FIG. 4 illustrates an end view of the lordotic spacer 10 (as seen from the distal end 12). As seen in FIG. 4, for the illustrated embodiment, the first side 16 is generally larger, that is, has a greater height, than the second side 18. For example, in the illustrated embodiment, the maximum height of the first side may be 16.4 millimeters, and the maximum height of the second side may about 15 millimeters. Further, the top curved surface 40 and the bottom curved surface 42 are also radiused as seen from the end. As discussed below, these radii (as viewed from the distal end) change along the length of the lordotic spacer. Because the first side 16 has a generally larger height than the second side 18, lines extending generally from the second side 18 to the first side 16 tangent to the radii at a given point along the length of the spacer may diverge at an angle 46. For example, in the illustrated embodiment, the angle 46 may about 10 degrees. As discussed above, the width of the spacer may be less than the height to facilitate insertion and rotation. For example, in the illustrated embodiment, the lordotic spacer 10 has a width (the distance between the first side 16 and the second side 18) of about 10 millimeters. As seen in FIG. 4, in the illustrated embodiment the first side 16 and second side 16 are substantially straight. In alternate embodiments, these surfaces may also be curved and/or contain cutouts and/or projections for grasping the spacer and/or facilitating the flow of bone graft material.

In alternate embodiments, differently sized lordotic spacers may be provided to accommodate differently sized patients and anatomies. Further, lordotic spacers may be provided as part of a set of differently sized spacers. For example, in certain embodiments, spacers are provided in sets of 3 or 4, covering a range of sizes. As an example, the smallest spacer in the set may have a maximum height at the second side of about 7 millimeters, and the largest spacer in the set may have a maximum height at the second side of about 15 millimeters height. Further, the smallest spacer in the set may have a width of about 7 millimeters, and the largest spacer in the set may have a width of about 10 millimeters.

As indicated above, the lateral curvature of the top and/or bottom surfaces (that is, the curvature of these surfaces from one side to another, or as viewed from the distal or proximal end) may vary along the length of the spacer. In the embodiment illustrated in FIGS. 1-7, the radius of curvature of the top surface 40 and the bottom surface 42 increases distally along the length of the spacer. Put another way, the radius of curvature of these surfaces generally increases toward the distal end 12, or these surfaces become flatter toward the distal end.

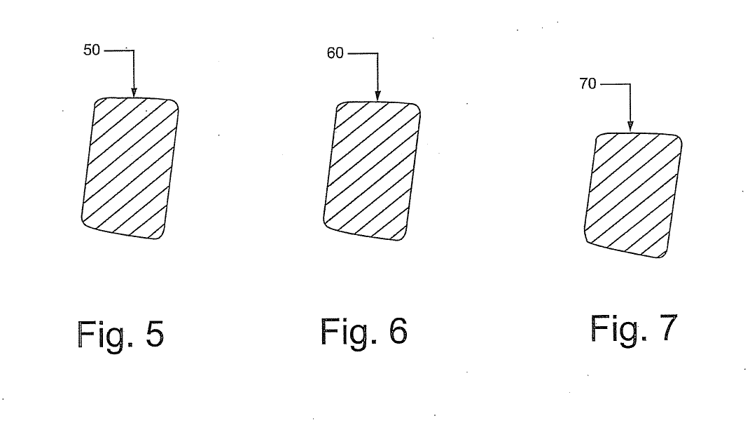

FIGS. 5-7 illustrate cross-sections taken at various points along the length of the lordotic spacer 10. FIG. 5 illustrates a cross-section taken along a plane extending generally perpendicularly through the lordotic spacer 10 along line 5-5, about 6 millimeters from the distal end 12 (or about 24 millimeters from the proximal end 14); FIG. 6 illustrates a cross-section taken along a plane through line 6-6, about 14.1 millimeters from the distal end 12 (or about 15.9 millimeters from the proximal end 14); and FIG. 7 illustrates a cross-section taken along a plane through line 7-7, about 24 millimeters from the distal end (or about 6 millimeters from the proximal end 14).

The top surface 40 includes a first lateral radius 50 along a plane through line 5-5 (see FIG. 5), a second lateral radius 60 along a plane through line 6-6 (see FIG. 6), and a third lateral radius 70 along a plane through line 7-7 (see FIG. 7.) In the illustrated embodiment, the first lateral radius 50 is about 55.7 millimeters, the second lateral radius 60 is about 44.5 millimeters, and the third lateral radius 70 is about 35.9 millimeters. Thus, the radius of curvature from side to side across the top surface 40 generally increases distally along the length of the lordotic spacer 10. Put another way, the lateral curvature becomes generally flatter toward the distal end 12 and generally less flat toward the proximal end 14. In the illustrated embodiment, the lateral radius changes substantially continuously and substantially smoothly along the length of the lordotic spacer. In other embodiments, the lateral radius may be changed in a series of discrete steps, and/or the lateral curvature may be formed by a series of angles and/or steps, or other suitable geometries (for example, portions curved similar to an oval). The center of curvature may be aligned along a central plane drawn equidistant between the two sides, or it may be offset from such a line. The maximum radius may be at or near the distal end, and the minimum radius may be at or near the proximal end. In the illustrated embodiment, the bottom surface 42 is curved substantially similarly to the top surface 40, with substantially the same lateral radius as the top surface 40 at any given location along the length of the lordotic spacer 10. In alternate embodiments, the bottom surface may be curved differently than the top surface.

As depicted in FIGS. 5-7, the lordotic spacer 10 is substantially solid along its length. In other embodiments, the spacer may contain hollow portions and/or channels or cutouts extending into and/or through the spacer. Also, in the illustrated embodiment, the lordotic spacer 10 has substantially straight sides. In alternate embodiments, however, the sides may be curved, tapered, and/or angled in one or more directions, and may also include cutouts and/or projections. Such cutouts or projections, for example, may be configured to help direct the flow of bone graft material, or to provide a location for bone graft material to be placed. Such cutout or projections may alternatively or additionally be configured to help facilitate grasping, manipulating, inserting, and/or rotating the spacer.

Generally speaking, the various radii and other proportions of the spacer are selected to mate as closely as possible with the shape of the vertebral surfaces to which the spacer will be adjacent or near (thereby reducing, minimizing, or eliminating the need to prepare the surfaces (such as, for example, by re-shaping the surfaces) and/or remove vertebral material prior to placing the spacer), as well as to maintain a desired spatial relationship and positioning (including the lordotic curvature) of the spine. As indicated above, different sizes of spacers may be used to accommodate different procedures and/or sizes of patient anatomy. The spacer may, for example, be made of PEEK (polyether ether ketone), titanium, carbon fiber, bone allograft, or a plurality of materials.

FIG. 8 illustrates a plan view of a lordotic spacer 100 in place after being inserted and positioned in a patient. The lordotic spacer 100 includes a distal end 102 and a proximal end 104, and a first side 106 and a second side 108. The lordotic spacer 100 may be substantially similar in many respects to the spacer described above. For example, in the illustrated embodiment, the first side 106 is positioned generally distally of the second side 108. The spacer 100 is at an angle 110 to a line 112 cutting through the spinal column anteriorly to posteriorly. For example, in the illustrated embodiment, the angle is about 45 degrees. The height of the spacer generally increases distally, and is generally larger toward the first side 106 than the second side 108. Thus, both of these general increases in height can help encourage the spine into a desired amount of posterior concave curvature (i.e. a concavity that faces the posterior of the patient).

In certain embodiments, the spacer may be inserted as follows. To insert the spacer 100, first an incision is made from the desired side and at the desired angle, and any necessary preparation of the vertebral surfaces performed. A properly sized spacer is selected, and inserted, distal end first, with the sides oriented vertically with respect to the patient (that is, at a ninety degree rotation from its final desired orientation). The spacer is advanced to its desired position, and then rotated to its final orientation, with the taller side generally distal of its shorter side. Finally, any desired bone graft material is introduced into the site of interest.

While particular embodiments of the invention have been shown, it will be understood that the invention is not limited thereto since modifications may be made by those skilled in the art, particularly in light of the foregoing teaching. It is therefore, the appended claims that define the true spirit and scope of the invention.

What is claimed is:

1. A spacer for maintaining the position of adjacent vertebrae, the spacer including:
a distal end and a proximal end, the distance between the distal end and the proximal end defining a length;
top and bottom surfaces spaced by first and second sides, the top and bottom surfaces defining a height, and the first and second sides defining a width, wherein the height of the spacer generally increases distally and generally increases from the second side to the first side;
wherein at least one of the top and bottom surfaces defines a curved surface, wherein a lateral curvature is defined as the curvature along the curved surface at a given point along the length of the spacer, taken along a plane extending between the first and second sides and extending generally perpendicular to the sides; and
wherein the radius of the lateral curvature generally increases distally along the length of the spacer.
